SEQUENCE LISTING (4 sequences)

SEQ ID NO: 1 - DNA, 28 bp, Artificial Sequence
  Primer
gggaattcatgggtgccccgacgttgcc

SEQ ID NO: 2 - DNA, 28 bp, Artificial Sequence
  Primer
ctctcgagtcaatccatggcagccagct

SEQ ID NO: 3 - DNA, 27 bp, Artificial Sequence
  Primer
attcgtccggcgccgctttcctttctg

SEQ ID NO: 4 - DNA, 27 bp, Artificial Sequence
  Primer
cagaaaggaaagcggcgccggacgaat

(19) United States
(12) Patent Application Publication
(10) Pub. No.: US 2007/0081978 A1
(43) Pub. Date: Apr. 12, 2007

(54) Adeno-associated virus-mediated survivin mutants and methods related thereto

(76) Inventors: Shui Ping Tu, New York, NY (US); Marie Chia-Mi Lin, Hong Kong (HK); Hsiang-Fu Kung, Hong Kong (HK); Benjamin Chun-Yu Wong, Hong Kong (HK)

Correspondence Address: COOPER & DUNHAM, LLP, 1185 AVENUE OF THE AMERICAS, NEW YORK, NY, 10036, US

(21) Appl. No.: 11/245,768

(22) Filed: Oct. 7, 2005

(51) Int. Cl. A61K 48/00; C12N 15/861

(52) U.S. Cl. 424/93.2; 435/456

(57) ABSTRACT

This invention provides two novel vectors, a vector comprising a rAAV-type 2 plasmid encoding mutant survivin (Cys84Ala), and a vector comprising a rAAV-type 2 plasmid encoding eGFP. This invention also provides compositions comprising the above vectors. This invention provides a method for inducing apoptosis in a cell comprising introducing into the cell the vector comprising a rAAV-type 2 plasmid encoding mutant survivin (Cys84Ala), or a composition thereof. This invention further provides a method for inhibiting tumor cell growth comprising introducing into the tumor cell the vector comprising a rAAV-type 2 plasmid encoding mutant survivin (Cys84Ala), or a composition thereof. Finally, this invention provides a method of treating a subject having colon cancer comprising administering to the subject a suitable amount of the vector comprising a rAAV-type 2 plasmid encoding mutant survivin (Cys84Ala) in a composition.

### BACKGROUND OF THE INVENTION

Survivin is a member of the inhibitor of apoptosis (IAP) gene family. It is expressed in G₂-M phase and its interaction with the mitotic spindle apparatus is essential for its anti-apoptotic function.¹⁻³ Overexpression of survivin inhibits apoptosis induced by various apoptotic stimuli in vitro and in vivo,⁴⁻⁶ initiates cell division by accelerating the entry into S phase,⁷ and promotes chemical-induced tumor progression.⁸ Survivin is expressed in most of the common human tumors, but not in normal adult differentiated tissues.^{1,4} Expression of survivin correlates with reduced apoptotic index, poor prognosis and increased risk of recurrence in cancer patients.⁹⁻¹² Furthermore, survivin has been implicated as a critical regulator of angiogenesis.¹³ Survivin expression protects proliferative endothelial cells from apoptosis, and mediates the ability of angiopoietin to stabilize vascular structures during angiogenesis.¹⁴⁻¹⁶ Since tumor development and growth depend on angiogenesis, which is in turn dependent on endothelial viability, targeting survivin might favor apoptotic involution of newly formed blood vessels and indirectly inhibit tumor formation, acting as a type of anti-angiogenic therapy.¹⁷ These results suggest that survivin may have significant potential as a new target in the treatment of cancer.

Previous studies have shown that targeting survivin resulted in spontaneous apoptosis, enhancement of chemotherapy-induced cell death and inhibition of tumor growth in vitro and in vivo.¹⁸⁻²³ However, these studies have used either plasmid or adenoviral vectors.^{24,25} The transfer of plasmid DNA is typically an inefficient process, and adenoviral-mediated gene transfer is complicated by a host immune response to the transduced target cells.²⁶ In addition, only transient transgene expression is achieved by these approaches. Consequently, after an initial delay, tumor growth often resumes with the loss of expression of the therapeutic gene.²⁶ Thus, long-term expression of therapeutic genes is likely to be required to achieve a sustained anticancer effect.

Adeno-associated virus vectors have been used widely to achieve efficient and long-term gene delivery in the treatment of numerous genetic diseases in a wide variety of animal models.^{27,28} as well as in preliminary human clinical trials.²⁹ AAV is the only viral vector system based on a nonpathogenic and replication-defective virus that can effectively transduce a broad range of host tissues, including both proliferating and post-mitotic cells.^{26,30-33} AAV vectors penetrate human solid tumor tissue in vivo more effectively than adenoviral vectors.³¹ As a result, AAV vectors may meditate gene transfer to malignant tumors with longer duration, greater safety, efficiency and consistency than any other available gene delivery system.

Survivin is overexpressed in 53%-64% of colorectal cancers,^{32,33} and its expression is correlated with an aggressive phenotype, angiogenesis, and overall poor survival in patients with colorectal cancer.³²⁻³⁵ Furthermore, oral administration of an AAV vector leads to persistent expression of the transgene in both gut epithelial and lamina propria cells.³⁶ These results indicate that AAV may have tropism for colon cancer cells. However, AAV viruses have not been studied in human colon cancer. In this study, rAAV-survivin mutant (Cys84Ala) was generated and the effect of AAV mediated survivin mutant (Cys84Ala) in the treatment of colon cancer was evaluated.

### SUMMARY OF THE INVENTION

This invention provides a vector comprising a rAAV-type 2 plasmid encoding mutant survivin (Cys84Ala). This invention also provides compositions comprising the above vector.

This invention provides a method for inducing apoptosis in a cell comprising introducing into the cell the vector comprising a rAAV-type 2 plasmid encoding eGFP, or a composition thereof.

This invention further provides a method for inhibiting tumor cell growth comprising introducing into the tumor cell the vector comprising a rAAV-type 2 plasmid encoding mutant survivin (Cys84Ala), or a composition thereof. This invention provides a method of treating a subject diagnosed with colon cancer comprising administering to the subject a suitable amount of the vector comprising a rAAV-type 2 plasmid encoding mutant survivin (Cys84Ala), or a composition thereof.

### BRIEF DESCRIPTION OF THE FIGURES

FIG. 1A-C

Effect of AAV-Sur-Mut(Cys84Ala) on cell proliferation. (A) SW1116 cells were infected with rAAV-eGFP with 1×10⁵ viral particles/cell. After 4 days, cells were observed by fluorescence microscopy, and photographs were taken from representative experiment. (B) SW1116 and Colo 205 cells were infected with rAAV-eGFP with 1×10⁵ viral particles/cell. After 4 days, cells were analyzed for eGFP expression using flow cytometry. The results represent the mean transduction efficiency from 3 parallel experiments. (C) SW1116 cells were plated into 96 well plates for 24 hours. Then cells were infected with indicated dose of rAAV for 96 hours. Cell proliferation was measured by MTT assay. The values were expressed as means±SEM from three independent experiments. *P<0.01 versus corresponding mock infection and rAAV-eGFP treated groups.

FIG. 2A-C

rAAV-Sur-Mut(Cys84Ala) induced apoptosis in colon cancer cells. (A) Cells were infected with rAAV at 1×10⁵ viral particles/cell. Cells were harvested and analyzed by FACS to determine the percentage of apoptosis 4 days after infection. The results represent the means±SEM from 3 independent experiments. **P<0.001, *P<0.05, compared with rAAv-eGFP treated group. (B) Infection of rAAV-Sur-Mut(Cys84Ala) induced expression of mutant survivin protein, caspase-3 and PARP cleavage, and released mitochondrial cytochrome c in SW1116 cells. Cells were infected with rAAV with 1×10⁵ viral particles/cell. Survivin, caspase-3, PARP, cytochrome c and β-actin proteins were detected by Western blot analysis. (C) Infection of rAAV-Sur-Mut(Cys84Ala) increased caspase-3 activity in SW1116 cells. Cells were infected with rAAV at 1×10⁵ viral particles/cell. Protease activities at each time point were assessed using the substrate DEVD-ρNA. Data are expressed as means±SD of 3 different experiments.

FIG. 3A-B

rAAV-Sur-Mut(Cys84Ala) transduction induces mitotic catastrophe in colon cancer cells. (A) SW1116 cells were transduced with rAAV-Sur(wt), rAAV-eGFP, or rAAV-Sur-Mut(Cys84Ala), or mock transduced with PBS. Seventy-two hours post-transduction, cells were stained for microtubules with an antibody to α-tubulin. Bold arrows showed abnormal large and multilobed nuclei. Photomicrographs are from representative experiments performed in duplicate. Original magnification ×400. (B) Approximately 500-600 nuclei were scored on 5 random 400× objective fields in duplicate as described. The experiment was performed independently and the results presented are the means±SEM obtained from three independent experiments. *P<0.001, compared with group transduced with PBS, rAAV-Sur(wt) and rAAV-eGFP.

FIG. 4A-B

rAAV-Sur-Mut(Cys84Ala) expression inhibits tumor formation. (A) Tumor growth curve. SW1116 cells were transduced with PBS, rAAV-Sur(wt), rAAV-eGFP or rAAV-Sur-Mut (Cys84Ala) for 12 hours. 2×10⁶ viable infected cells were injected into the right flank. Tumors were monitored every three days. Each point represents the mean tumor size (as measured by three perpendicular diameters). Each bar represented the mean tumor size with 95% confidence intervals for four mice. *P<0.05, compared with those transduced with rAAV-Sur(wt) and PBS. **P<0.001, compared with those transduced with rAAV-eGFP. (B) Inhibition of survivin function induced apoptosis in vivo. Tumor sections were subjected to immunohistochemical staining for survivin and TUNEL stained for apoptosis (apoptotic cells are green). (Original magnification ×200)

FIG. 5A-C

rAAV-Sur-Mut(Cys84Ala) inhibited tumor growth. (A) Untreated SW1116 cell were subcutaneously injected into the right flank of athymic female nude mice. Tumors were allowed to grow to approximately 100-150 mm³ volume. Masses were injected in three sites with rAAV-Sur-Mut(Cys84Ala), rAAV-eGFP or rAAV-Sur(wt) at 5×10¹⁰ viral particles/site of injection, or with PBS. Photographs were taken from representative mice 4 weeks after treatment. (B) Tumor growth was measured every week after injection. Data are the means±SEM of tumor size per mouse. (C) Tissue sections of formalin-fixed, paraffin-embedded xenograft tumors were stained by H&E, immunohistochemical stained for survivin or TUNEL stained for detection of apoptotic cells. Images were representative fields of view from tumors 5 days after treatment. (Original magnification ×200)

FIG. 6A-B

rAAV-Sur-Mut(Cys84Ala) prolonged the survival of mice. (A) Masses were injected in three sites with rAAV-Sur-Mut(Cys84Ala), rAAV-Sur(wt) and rAAV-eGFP at 5×10¹⁰ viral particles/site of injection, or in combination with 5-Fluorouracil for 5 days. Tumor xenografts were collected and tissue sections of formalin-fixed, paraffin-embedded xenograft were TUNEL stained for apoptosis. (B) Survival curve. The experimental conditions were as in (A) Survival was monitored every day, and tumor volume was measured every week after treatment. Definition of death is natural death due to tumor burden or killing due to tumor sizes (diameter) more than 2.5 cm.

FIG. 7A-B

Immunofluorescent staining for CD-31/PECAM-1 and apoptosis of endothelial cells. (A) Tumor angiogenesis was assessed by immunofluorescent staining with CD-31 (endothelial cells) on frozen sections of tumors injected with rAAV-Sur-Mut(Cys84Ala), rAAV-Sur(wt) and rAAV-eGFP virus and PBS (upper panel). Apoptosis of endothelial cells was assessed by sequential immunofluorescent staining for CD31 and TUNEL performed in tumor xenografts (middle and lower panels). Apoptotic cells stain green, whereas endothelial cells stain red. When endothelial cells undergo apoptosis, the overlay of green and red yields yellow staining. Bold arrow shows apoptotic endothelial cells. (Original magnification ×200) (B) Quantification of angiogenesis and apoptosis of endothelial cells, performed as described in Material and Methods. The results are the mean of independent determinations of two investigators. Data were shown as the mean±SD (error bars) of the number of five independent areas. **P<0.01.

### DETAILED DESCRIPTION OF THE INVENTION

This invention provides a novel vectors comprising an rAAV-type 2 plasmid encoding mutant survivin (Cys84Ala) and an rAAV-type 2 plasmid encoding eGFP. This invention also provides compositions comprising these vectors.

This invention further provides a method for inducing apoptosis in a cell comprising introducing into the cell the vector comprising an rAAV-type 2 plasmid encoding mutant survivin (Cys84Ala), or an rAAV-type 2 plasmid encoding eGFP, or a composition thereof. In one embodiment, the cell is a cancer cell. In a further embodiment, the cancer cell is a colon cancer cell. In another embodiment, the vector is introduced into the cell by transduction.

This invention further provides a method for inhibiting tumor cell growth comprising introducing into the tumor cell the vector comprising a rAAV-type 2 plasmid encoding mutant survivin (Cys84Ala), or a composition thereof. In one embodiment, the tumor cell is a colon cancer cell. In another embodiment, the vector is introduced into the cell by transduction.

This invention provides a method of treating a subject diagnosed with colon cancer comprising administering to the subject a suitable amount of the vector comprising a rAAV-type 2 plasmid encoding mutant survivin (Cys84Ala), or a composition thereof. In one embodiment, the composition comprises a suitable amount of the vector comprising a rAAV-type 2 plasmid encoding mutant survivin (Cys84Ala) together with a suitable chemotherapy agent. In another embodiment, the suitable chemotherapy agent is 5-FU or cisplatin.

As used herein, “administering” can be effected or performed using any of the various methods and delivery systems known to those skilled in the art. The administering can be performed, for example, intravenously, via cerebrospinal fluid, orally, nasally, via implant, transmucosally, transdermally, intramuscularly, and subcutaneously.

The following delivery systems, which employ a number of routinely used pharmaceutical carriers, are only representative of the many embodiments envisioned for administering the compositions of the present invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Experimental Details

Synopsis

Reactivation of survivin expression is involved in carcinogenesis and angiogenesis in colon cancer. Previous in vitro studies showed that mutation of the cysteine residue at position 84 (Cys84Ala) of survivin generates a dominant-negative mutant that triggers mitotic catastrophe and apoptosis. We investigated the therapeutic effect of adeno-associated virus (AAV) mediated survivin mutant (Cys84Ala) on colon cancer. Survivin mutant (Cys84Ala) (Sur-Mut(Cys84Ala)) was cloned into the AAV expression vector pAM/CAG-WPRE.poly(A), to generate recombinant AAV-Sur-Mut(Cys84Ala) virus. Cell proliferation, apoptosis, mitotic catastrophe, angiogenesis and tumor growth were measured in vitro and in vivo. Transduction of colon cancer cells with rAAV-Sur-Mut(Cys84Ala) inhibited cell proliferation and induced apoptosis and mitotic catastrophe in vitro. rAAV-Sur-Mut(Cys84Ala) sensitized colon cancer cells to chemotherapeutic drugs. Furthermore, expression of survivin mutant mediated by AAV inhibited tumorigenesis in colon cancer cells. Intratumoral injection of rAAV-Sur-Mut(Cys84Ala) significantly induced apoptosis and mitotic catastrophe, inhibited angiogenesis and tumor growth in a colon cancer xenograft model in vivo. More importantly, rAAV-Sur-Mut(Cys84Ala) expression strongly enhanced the antitumor activity of 5-Fluorouracil(5-FU), resulting in complete eradication of established tumors. These results showed that rAAV-Sur-Mut(Cys84Ala) induced apoptosis and mitotic catastrophe, inhibited tumor angiogenesis and tumor growth.

Materials and Methods

Cell Culture

Human colon cancer cell lines SW1116, Colo 205, HT-29 and SW147 (ATCC, Rockville, Md., USA) were maintained in RPMI-1640 containing 10% fetal bovine serum (FBS), 100 U/ml penicillin, and 100 μg/ml streptomycin (Gibco BRL, Life Technologies, NY, USA). Hydroxycamptothecin (HCPT, 5 μg/ml, WangSi Pharmaceutical Corp, WangSi, China) arsenic trioxide (AS₂O₃, 0.5 μM, Sigma, St. Louis, Mo.) cisplatin (2 μg/ml) and 5-fluorouracil (10 μg/ml, Pharmacia & Upjohn Limited Corp, Australia) were dissolved in DMSO and stored at 4° C. Human embryonic kidney cells (HEK293) were obtained from American Type Culture Collection. Cells were maintained at 37° C. in a humidified atmosphere of 5% CO₂ in the appropriate growth medium with supplements added as recommended. The 293 cell line was used for the construction and amplification of AAV vectors.

Construction of Survivin-sense and Dominant-negative Mutant Plasmids

The survivin gene was cloned by reverse transcriptase-polymerase chain reaction (RT-PCR). Total RNA was extracted from SW1116 cells using Trizol reagent (GIBCO BRL). cDNA corresponding to the coding region was generated by RT-PCR using the survivin forward primer: 5′-GGGAATTCATGGGTGCCCCGACGTTGCC-3′, reverse primer: 5′-CTCTCGAG TCAATCCATGGCAGCCAGCT-3′ (Genset Singapore Biothech. Pte Ltd, Singapore). The PCR product was inserted into the EcoR I and Xho I sites of pcDNA3(+) (Invitrogen, Groningen, Netherlands ) to generate pcDNA3-Surivin.

An overlap extension PCR³⁷ was used to construct a dominant-negative mutant of survivin, using pcDNA3-survivin plasmid as a template. The 5′-flanking forward primer and 3′-flanking reverse primer were described as above. For survivin Cys84Ala mutant, we employed the forward primer 5′-ATTCGTCCGGC GCCGCTTTCCTTTCTG-3′, and the reverse primers 5-CAGAAAGGAAAGCGGCGCCGGACGAAT-3′, which produced a TGC-to-GCC substitution at nucleotides 251-253, resulting in the encoding of an alanine residue at amino acid 84 rather than the wild-type cysteine. The fragment containing the mutant survivin was cloned in the EcoRI and XhoI sites of the pcDNA3 vector, generating the pcDNA3-Sur-Mut (Cys84Ala) plasmid. All constructs were confirmed by sequencing.

Construction and Generation of Recombinant AAV

Two recombinant AAV type 2 plasmids³⁸ encoding mutant survivin (Cys84Ala) and eGFP, respectively, were constructed. The recombinant AAV2 packaging plasmid pAM/CAG-WPRE-BGH-polyA, was constructed by deleting all of the viral open reading frame (ORF). The chicken β-actin promoter and cytomegalovirus (CMV) enhancer, a multicloning site, and the bovine growth hormone polyadenylation signal were inserted between the flanking 145 base pair inverted terminal repeats (ITRs) required in cis for replication of the viral genome. Full-length Sur(wt) and Sur-Mut(Cys84Ala) cDNAs were cut with BamHI and XhoI from pcDNA3-Survivin and pcDNA3-Sur-Mut(Cys84Ala) respectively, and subcloned into the corresponding BamHI and XhoI sites of pAM/CAG to generate pAM/CAG-Sur-Mut (Cys84Ala). The 785 bp eGFP cassette was removed from pAV/NSE-eGFP-WPRE.poly(A) (38) with XhoI and EcoRI and subcloned into the XhoI and EcoRI sites of pAM/CAG to generate pAM/CAG-eGFP. Helper-free recombinant AAV stocks were generated by calcium phosphate cotransfection of the vector plasmids with the packaging plasmid pDG in 293 cells to obtain rAAV-Sur-Mut(Cys84Ala), rAAV-Sur(wt) and rAAV-GFP virus. Cells were harvested between 65 and 70 h post-transfection and lysed in 0.5% deoxycholate (Fisher Scientific) in the presence of 50 units/ml Benzonase (Sigma, St. Louis, Mo.) for 30 min at 37° C. After two cycles of freezing and thawing followed by centrifugation, supernatants containing AAV vectors were combined and purified by HiTrap Heparin column chromatography (Sigma, St. Louis, Mo., USA). Peak virus fractions were collected and dialyzed against PBS supplemented with 1 mM MgSO₄, and then concentrated using a 100 K-MicroSep centrifugal concentrator (Life technologies, Carlsbad, Calif., USA). The AAV viral genome titer was quantified by Real-Time PCR using TaqMan (Perkin-Elmer Biosystems, Foster City, Calif., USA). Recombinant AAV-eGFP was generated by the same procedure. Viral titers were determined to be 1˜5×10¹² particles/ml by comparing band intensities in the control lane to those of rAAV-Sur-Mut(Cys84Ala), rAAV-Sur(wt) and rAAV-GFP.

In Vitro Transduction

Cells (2×10⁵/well for six-well plates, 5×10⁴/well for 24-well plates, and 5×10³/well for 96-well plates) were transduced with AAV virus by directly applying the diluted viruses into the growth medium. Cells were infected with the various viruses at the indicated multiplicity of infection. HEK 293, known to be highly permissive for AAV infection, was used as a reference cell line to optimize in vitro conditions. In all in vitro experiments, cells were treated with 500 μM tyrphostin 1 (Sigma Chemical Co., St. Louis, Mo.) for 2 h at 37° C. prior to infection. Tyrphostin 1 is a fibroblast growth factor receptor protein tyrosine kinase, known to augment AAV-mediated transgene expression in vitro.³⁹ After treatment, cells were washed twice with PBS and were either mock infected or infected with 1×10⁴ to 2×10⁵ viral particles/cell of the rAAV-Sur-Mut(Cys84Ala), rAAV-Sur(wt) and rAAV-GFP virus in DMEM supplemented with 10% FBS. Cultures were analyzed by FACS and fluorescence microscopy to determine the transduction efficiency.

MTT assay

Cell proliferation was measured by MTT assay. Exponentially growing cells were plated on 96 well plates at a density of 5000 cells/well and allowed to grow for 24 hours. Cells were then transduced with varying concentrations of rAAV virus for 96 hours. One hundred microliters of MTT stock solution (1 mg/ml) was added into each well, and cells were further incubated at 37° C. for 4 hours. Supernatants were removed and isopropanol used to dissolve the formazan product. The absorbance at wavelength 595 nm was measured in a micro-ELISA reader (Bio-Rad, Hercules, Calif.). The ratio of the absorbance of treated cells relative to that of the control cells were calculated and expressed as percentage of growth inhibition.

Flow Cytometry

Cells were collected and fixed in ice-cold 70% ethanol in PBS and stored at −20° C. before use. After re-suspension, cells were washed and incubated with 100 μl of RNase I (1 mg/ml) and 100 μl of propidium iodide (400 μg/ml; Sigma) at 37° C. for 30 min. Samples were analyzed by flow cytometry (Coulter, Luton). The cell-cycle phase distribution was calculated from the resultant DNA histogram using Multicycle AV software (Phoenix Flow Systems, San Diego, Calif.). Cells with subdiploid DNA content were scored as apoptotic.

Western Blot Analysis

Cells were disrupted in lysis buffer (50 mM Tris-HCl, pH 7.5, 250 mM NaCl, 0.1% NP-40, and 5 mM EGTA containing 50 mM sodium fluoride, 60 mM β-glycerol-phosphate, 0.5 mM sodium-vanadate, 0.1 mM PMSF, 10 μg/ml aprotinin, and 10 μg/ml leupeptin). Protein extracts were electrophoresed on 10% denaturing sodium dodecylsulfate gels, and transferred to Immobilon-P membranes (Millipore, Bedford, Mass.). The blots were incubated with specific primary antibodies followed by a peroxidase-conjugated second antibody (Santa Cruz, Calif.) and visualized by enhanced chemiluminescence (ECL, Amersham, Piscataway, N.J.). Survivin (2 μg/ml) polyclonal antibody was purchased from Alpha Diagnostic International Inc (San Antonio, Tex.), poly-(ADP-ribose)-polymerase (PARP), Monoclonal antibodies against β-actin, caspase-3 and cytochrome c were all purchased from Santa Cruz (Santa Cruz, Calif.).

Caspase-3 Activity Assay

Caspase-3 activity was determined using the ApoAlert Caspase Colorimetric Assay Kit according to the manufacturer's instructions (Clontech, Palo Alto, Calif.). Briefly, cells were lysed in the manufacturer's lysis buffer on ice for 10 min. Extracts were thawed and diluted with an equal volume of 2× reaction buffer containing DTT (10 mM) and the colorimetric caspase-3 substrate (DEVD-ρNA). Mixtures were maintained at 37° C. for 45 min and then analyzed in a spectrophotometer at 405 nm. Caspase-3 activity was calculated in pmol/min according to the manufacturer's instructions.

Immunofluorescence

Cells were grown on 2 well glass chamber slides (Nunc, Naperville, Ill.) and transfected with plasmids as described previously. Cells were fixed in 2% formaldehyde for 10 min and permeabilized with 0.5% Nonidet P40 in PBS. α-tubulin antibody (clone DM1A, diluted 1:100), and FITC-conjugated goat anti-mouse antibody were purchased from Sigma (St.Louis, Mo.). Nuclei were stained with 1 μg/ml Hoechst 33452 and cells were analyzed using a Zeiss Axioscop fluorescence microscope.

Immunohistochemistry

Survivin expression in tumor tissues was detected using a DAKO LSAB+ (Labelled streptavidin-biotin) Kit (DAKO, Carpenteria, Calif.) according to the manufacturer's instructions. Briefly, slides were boiled in 10 mM citrate buffer, pH 6 (Bio Genex, San Ranmon, Calif.) for antigen retrieval, and incubated with anti-survivin polyclonal antibody (1:100, Alpha Diagnostic International Inc, San Antonio, Tex.), followed by biotinylated anti-IgG antibody and streptAB-complex/HRP (DAKO, Carpentaria, Calif.). Sections were counterstained with hematoxylin and were independently evaluated by two blinded investigators. Survivin staining was recorded as the ratio of positively stained cells to all tumor cells in 5 random areas at 200-fold magnification.

In Situ Detection of Apoptotic Cells by TUNEL Assay

Xenograft tumors were excised and immediately fixed in formalin. TUNEL (terminal deoxynucleotidyltransferase-mediated deoxyuridine triphosphate-digoxigenin nick end labeling) staining was carried out using the ApoAlert DNA Fragmentation Assay Kit (Cat. No. K2024-1, Clontech Corp. Palo Alto, Calif.) according to the manufacturer's instructions. The percentage of apoptotic cells was assessed in 10 randomly selected fields viewed at 400× magnification. The apoptotic index (A/I) was calculated as number of apoptotic cells/total number of nucleated cells ×100%.

Immunofluoresence Double Staining of Endothelial Cells and Apoptotic Cells

Frozen tissues were sliced in 8 μm sections, mounted on positively charged slides (Fisher Scientific), air-dried for 30 minutes, and then fixed in cold acetone for 5 minutes. Slides were incubated with a protein-blocking solution for 20 minutes, and then incubated with rat monoclonal antimouse CD31 antibody (Pharmingen, San Diego, Calif., #550274) for 18 hours at 4° C. After PBS washing, the slides were incubated with a Texas Red fluorescently labeled anti-rat secondary antibody (1:200; Jackson ImmunoResearch Laboratory, Inc., West Grove, Pa.) for 1 hour in the dark at room temperature. Sections were washed twice with PBS and TUNEL stained (ApoAlert DNA Fragmentation Assay Kit, Clontech) according to the manufacturer's instructions. The slides were examined under an immunofluorescent microscopy (Zeiss Plan-Neofluor; Carl Zeiss, Thomwood, N.Y.) with a 100× objective, and images were captured using a digital camera (Photometrics, Tucson, Ariz.). Images were further processed with Adobe Photoshop software (Adobe Systems, Mountain View, Calif.). For quantification of endothelial cells, the slides were incubated with 300 μg/mL Hoechst stain for 10 minutes. CD31-positive endothelial cells were identified by red fluorescence and apoptotic cells exhibited nuclear green fluorescence. Endothelial cells undergoing apoptosis fluoresced yellow due to the overlapping green and red emissions. Microvessel density (MVD) was evaluated according to previously described method.^{40,41} Angiogenesis scores in each sample were expressed as an average of the absolute number of vessels in 5 random fields of view at 200× magnification. Percentage of apoptotic endothelial cells was expressed as an average of the ratio of apoptotic endothelial cells to the total number of endothelial cells in 5 random 0.01 1-mm² fields at 400× magnification.

Experimental Animal Model and Tumorigenic Assay

Female BALB/c nude mice, 5-6 weeks old, were bred in the Animal Laboratory Unit, The University of Hong Kong, Hong Kong. Institution guidelines were followed in handling the animals. Colon cancer cells were transduced in vitro at 5×10⁴ viral particles/cell with rAAV-Sur-Mut(Cys84Ala), rAAV-Sur(wt) and rAAV-eGFP, or mock infected with PBS. Twelve hours after transduction, cells were counted, and 2×10⁶ SW1116 viable cells were injected into the right and left flank of 6-week-old female nude mice respectively, four mice per group. All experiments were repeated at least twice. Tumor size was determined by measuring two perpendicular diameters with a caliper every three days. Tumor volume (V) was estimated by using the equation V=4/3π×L/2×(W/2)², where L is the mid-axis length and W is the mid-axis width. Some animals were sacrificed when the xenograft reached 1.0 cm. diameter for histology. Tumors tissues were removed, formalin fixed, embedded in paraffin, and stained with H&E, TUNEL and immunochemical staining. The protocol was approved by the Committee on the Use of Live Animals in Teaching and Research, University of Hong Kong, Hong Kong.

AAV Mediated Survivin Mutant Gene Therapy in the Xenograft Model of Colon Cancer

Five- to six-week-old female BALB/c nude mice were injected subcutaneously on the flanks with 2×10⁶ exponentially growing SW1116 cells. Tumors were allowed to grow to 100˜150 mm³ (5˜7 mm diameter). For local administration of rAAV, masses were injected in three sites with rAAV-Sur-Mut(Cys84Ala), rAAV-Sur(wt) or rAAV-EGFP at 5×10¹⁰ viral particles/site of injection, or at each of the three site with PBS. Alternatively, mice were i.p. injected with 50 mg/kg 5-fluorouracil daily for 5 days, or a combination of rAAV virus and 5-Fluorouracil. Tumor growth was measured weekly after injection. In other experiments, mice received an injection of uninfected exponentially growing 2×10⁶ SW1116 cells. After 7 days, mice were given an injection of same dosage of rAAV virus and 5-FU (3 animals/group). After 5 days, animals were euthanized, and their total tumor burden was excised, fixed and embedded in paraffin blocks for H&E, TUNEL and immunohistochemical staining, or tumors were snap-frozen in liquid nitrogen for CD31 staining.

Statistical Analyses

Data are reported as means±SE. Statistically significant differences (P<0.05) between groups were detected using unpaired t test, paired t test, and nonparametric tests, such as the Wilcoxon signed rank test and the Mann-Whitney Rank Sum test, where appropriate for the data. For categorical data, we used the χ² test.

Results

Effect of rAAV-Sur-Mut(Cys84Ala) Infection on Proliferation in Colon Cancer Cells

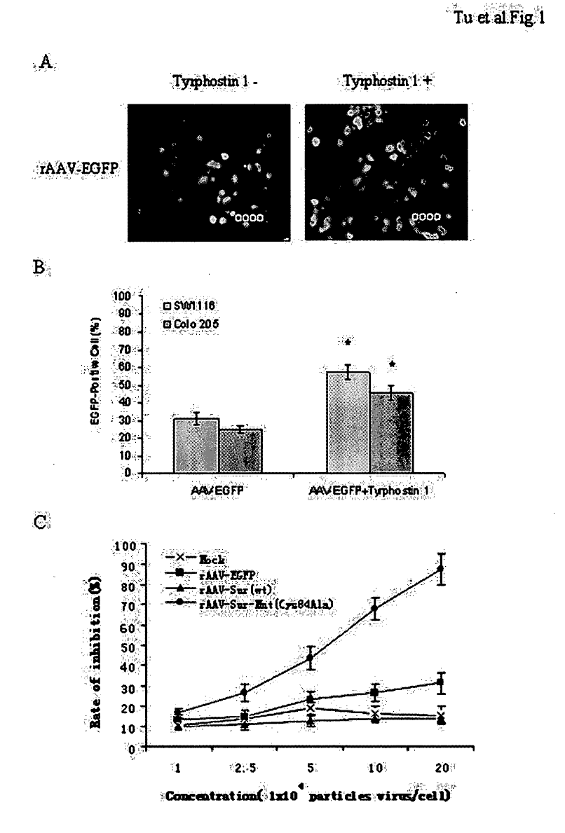

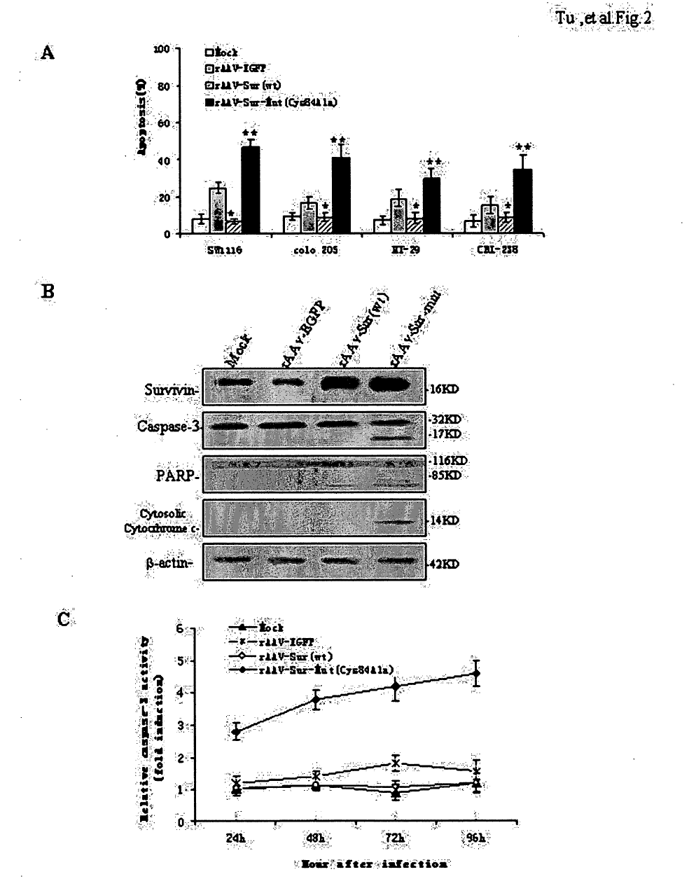

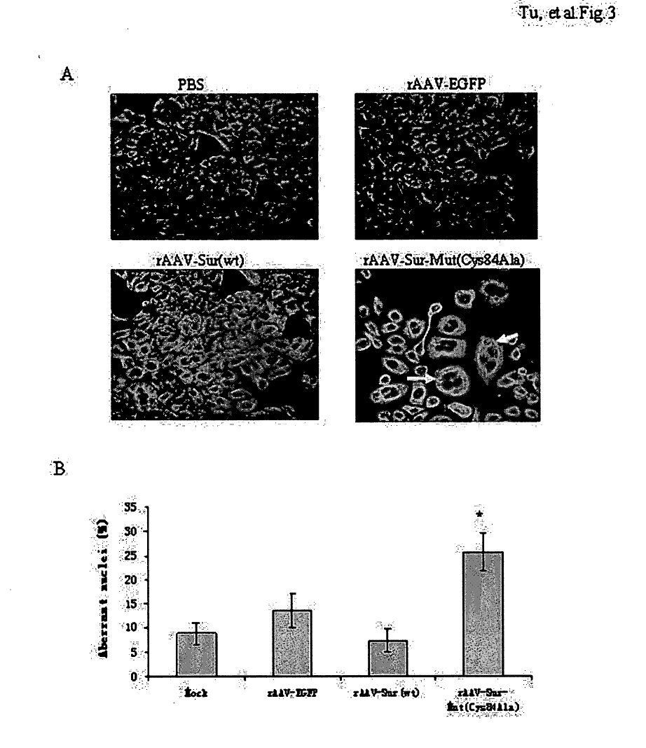

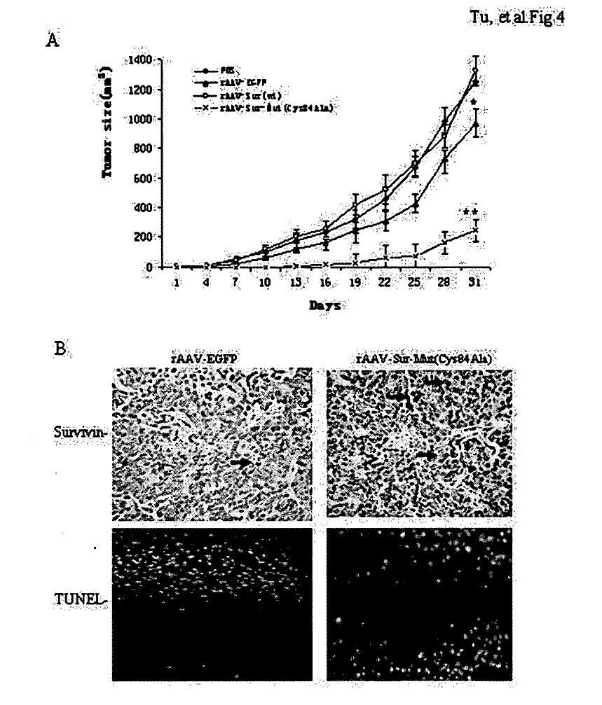

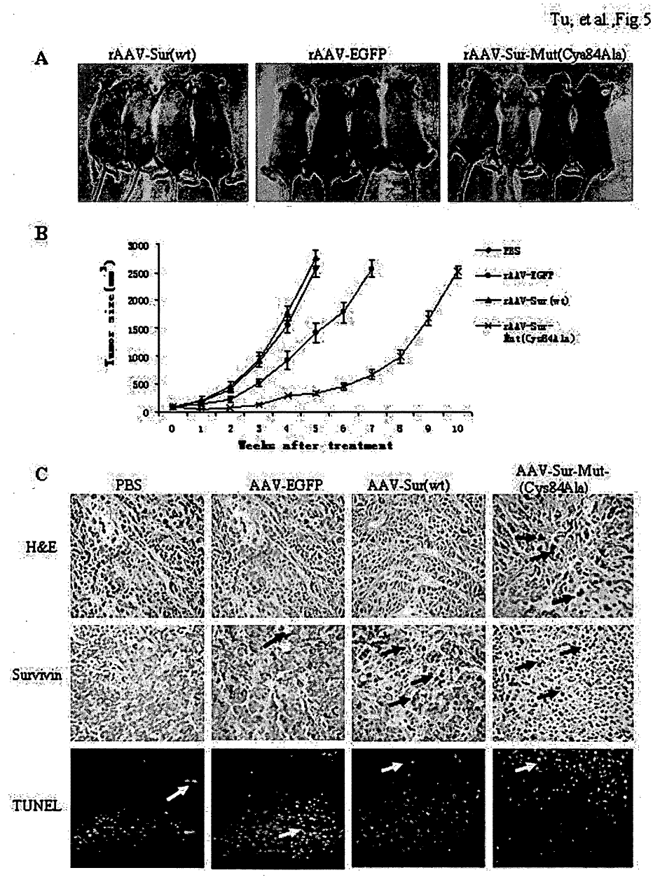

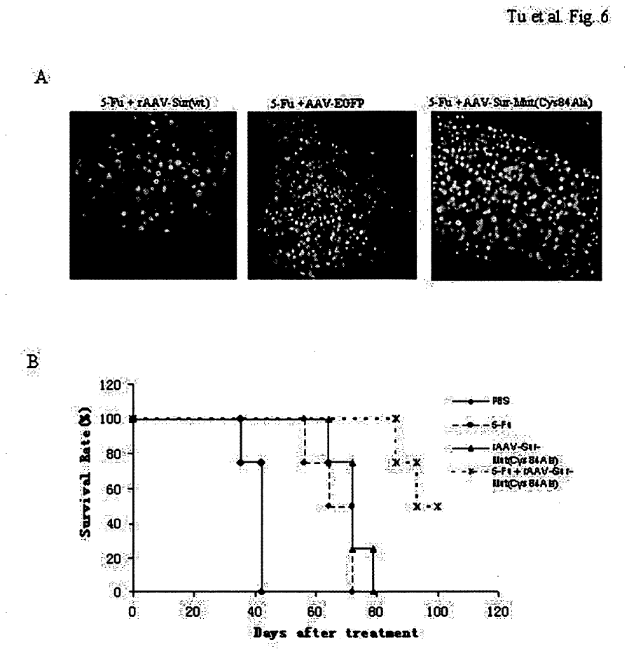

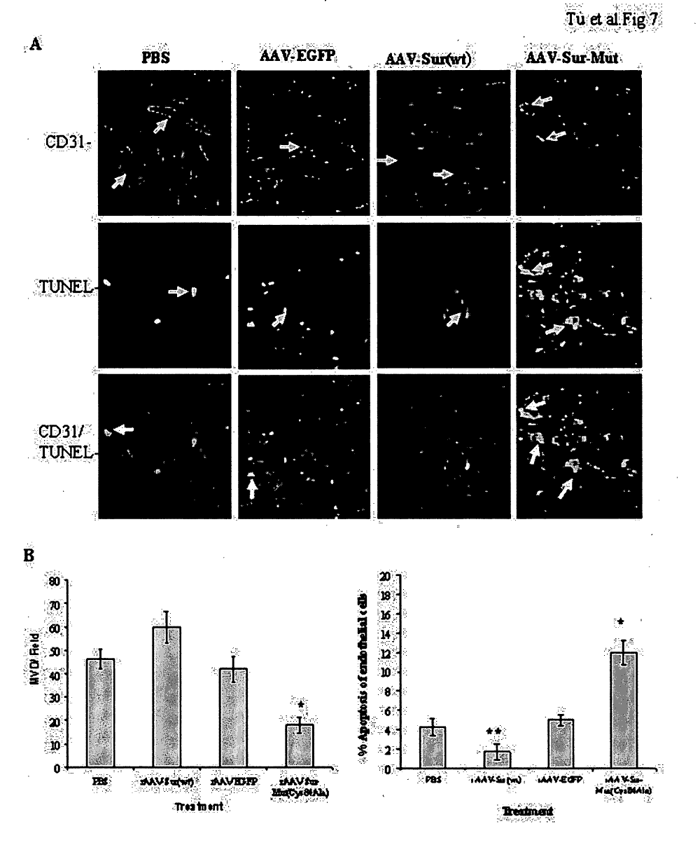

To determine the efficiency of viral transduction using the rAAV vectors, quantitative analysis was performed in different human colon cancer cell lines (SW1116, Colo 205, HT-29). Cells were plated in triplicate at a density of 5×10⁴ cells/well in 24-well culture plates and infected with rAAV-GFP (1×10⁵ viral particles/cell). 70%˜80% of HEK-293 cells were transduced by rAAV-eGFP as visualized in vitro by fluorescence microscopy and by the fraction of GFP-expressing cells measured by FACS analysis. The colon cancer cell lines were also efficiently transduced at rates ranging between 23%˜28%. Using a combination of rAAV and tyrphostin 1, the transduction rate was significantly enhanced to 48%˜57% (FIG. 1A and B).

The in vitro effect of rAAV-Sur-Mut(Cys84Ala) on cell proliferation was first evaluated. MTT assays demonstrated that infection with rAAV-Sur-Mut(Cys84Ala) caused a dosage-dependent increase in cell death, compared with rAAV-Sur(wt), rAAV-eGFP and mock infected cells (FIG. 1C). To exclude the possibility that preincubation with tyrphostin 1 affected cell survival, colon cells were infected with rAAV-eGFP or rAAV-Sur-Mut (Cys84Ala) with and without tyrphostin 1. Preincubation with tyrphostin 1, which led to accelerate transcription of the transgene, increased the percentage of dead cells after rAAV-Sur-Mut(Cys84Ala) transduction but did not affect mock, rAAV-Sur(wt) and rAAV-eGFP transduced cells (data not shown).

rAAV-Sur-Mut(Cys84Ala) Infection Induces Apoptosis in Colon Cancer Cells

To further characterize the pro-apoptotic effects of rAAV-Sur-Mut(Cys84Ala), apoptosis induced by transduction with rAAV-Sur-Mut(Cys84Ala), rAAV-Sur(wt) and rAAV-eGFP or PBS (mock infection) was analyzed. Transduction of rAAV-Sur-Mut(Cys84Ala) induced apoptosis in a time-dependent manner in all 4 cells lines (data not shown). The percent of apoptotic cells, as measured by flow cytometry, ranged from 20% to 43%. Representative examples of the analysis are shown in FIG. 2A. Transduction with rAAV-Sur-Mut(Cys84Ala) consistently resulted in expression of mutant survivin protein and several markers of apoptosis, such as caspase-3 and PARP cleavage, and the release of mitochondrial cytochrome C into the cytosol (FIG. 2B). Survivin expression levels detected by immunoblotting were assumed to reflect the cumulative levels of endogenous wild-type survivin and overexpressed dominant-negative mutant survivin (FIG. 2B). In contrast, there was no significant change in expression of other IAP genes (XIAP, cIAP1, cIAP2) in cells transfected with rAAV-Sur-Mut(Cys84Ala) (data not shown).

It was then investigated whether apoptosis induced by inhibition of survivin was mediated by caspase-3 activation. Our results showed that caspase-3 activity was significantly increased in SW1116 cells after transduction with rAAV-Sur-Mut(Cys84Ala), compared with cells transfected with control virus (eGFP) or virus encoding wild-type survivin (FIG. 2C).

Inhibition of Survivin Protein Function Using rAAV-Sur-Mut(Cys84Ala) Causes Mitotic Catastrophe in Gastric Cancer Cells

It was next determined whether transduction with rAAV-Sur-Mut(Cys84Ala) was sufficient to cause mitotic catastrophe in colon cancer cell lines. SW1116 cells infected with rAAV-Sur-Mut (Cys84Ala) revealed a statistically significant increase in abnormal nuclei that included micronucleated, multilobulated, and abnormally large nuclei relative to cells transduced with rAAV-Sur(wt), rAAV-eGFP, or mock transduced (FIG. 3A). The percentage of aberrant nuclei induced by rAAV-Sur-Mut(Cys84Ala) transduction and controls are summarized in FIG. 3B.

Infection of rAAV-Sur-Mut(Cys84Ala) Sensitizes Colon Cancer Cells to Chemo-Therapeutic Drugs

Colon cancer cell survival in cells transduced with rAAV-Sur-Mut(Cys84Ala) in combination with established anticancer agents was then examined. The rate of apoptosis was increased two to three fold when a combination of 5-FU and survivin dominant-negative virus was assessed relative to either treatment group alone (Table 1). Similar results were obtained when rAAV-Sur-Mut(Cys84Ala) was used alone or in combination with cisplatin (data not shown). In contrast, transduction with rAAV-Sur(wt) counteracted apoptosis induced by 5-FU.

TABLE 1
rAAV-Sur-Mut(Cys84Ala) sensetises colon
cancer cells to drug-induced apoptosis

| No. | Treatment group | % Apoptosis | P |
|---|---|---|---|
| 1 | 5-FU | 26.0 ± 3.4 | |
| 2 | rAAV-Sur(wt) | 9.4 ± 2.3 | |
| 3 | rAAV-EGFP | 14.1 ± 2.7 | |
| 4 | rAAV-Sur-Mut(Cys84Ala) | 25.4 ± 3.8 | 4 vs 2: P = 0.0070 |
| 5 | 5-FU + rAAV-Sur(wt) | 15.6 ± 2.4 | 5 vs 1: P = 0.0233 |
| 6 | 5-FU + rAAV-EGFP | 31.6 ± 5.8 | 6 vs 1: P = 0.3018 |
| 7 | 5-FU + rAAV-Sur-Mut | 58.4 ± 6.1 | 7 vs 1: P = 0.0028 |
| | (Cys84Ala) | | |

SW1116 cells were infected with rAAV at 1 × 10⁵ viral particle/cell, or followed by 10 μg/ml 5-fluorouracil for an additional 72 hour. Apoptosis was determined by FACS analysis. The results represented the mean ± SEM of three independent experiments. Mean percentage of apoptosis and P values between the different treatment groups 1-7 are indicated.

Adeno-Associated Virus Mediated Expression of Mutant Survivin Inhibits Tumor Formation

It was next explored whether transient expression of Sur-Mut(Cys84Ala) protein mediated by the rAAV vector could inhibit tumorigenesis. Expression of AAV encoding Sur-Mut(Cys84Ala) was confirmed by immunoblot analysis 24 hours after transduction with rAAV-Sur-Mut(Cys84Ala) (data not shown). Ex vivo transduction of SW1116 cells with rAAV-Sur-Mut(Cys84Ala) before injection into nude mice increased the latency period of the tumors and slowed the rate of tumor growth, whereas untransduced cells and cells infected with control viruses always produced tumors of comparable size (FIG. 4A). Interesting, mice injected with cells transduced with rAAV-Sur(wt) formed larger tumors than those injected with cells transduced with rAAV-eGFP (FIG. 4A). In two different experiments, 8 animals were injected with SW1116 infected with rAAV-Sur-Mut(Cys84Ala), with 4 of the 8 animals remained tumor free for more than 3 months. Inhibition of tumor formation coincided with expression of Sur-Mut(Cys84Ala) protein in tumor tissues (FIG. 4B). These data suggested that AAV-mediated expression of mutant survivin directly suppressed tumorigenicity.

rAAV-Sur-Mut(Cys84Ala) Inhibits Tumor Growth in vivo

Finally, the use of rAAV-Sur-Mut(Cys84Ala) as a potential treatment for established subcutaneous tumors was investigated. Uninfected SW1116 cells were injected into the flank of athymic nude mice, and tumors were allowed to reach a volume of approximately 100˜150 mm³ (5˜7 mm diameter). These tumors were injected in three sites with rAAV-Sur(wt), rAAV-eGFP or rAAV-Sur-Mut(Cys84Ala) at 5×10¹⁰ viral particles/site. Single intratumor administration of rAAV-Sur-Mut(Cys84Ala) inhibited tumor growth by approximately 81.5% at 4 weeks post-injection, while treatment with rAAV-eGFP only reduced tumor growth by 37.1%, respectively (FIG. 5A and B). Single intratumor administration of rAAV-Sur(wt) increased tumor growth by approximately 13.5% at 4 weeks after injection, compared with groups injected with PBS (FIG. 5A and B).

Tumors injected with rAAV-Sur-Mut(Cys84Ala) expressed high levels of mutant survivin protein, contained more apoptotic cells, and exhibited a higher frequency of cells with abnormally large nuclei (FIG. 5C). In contrast, tumors injected with rAAV-eGFP exhibited fewer TUNEL-positive cells (FIG. 5C). Tumors injected with rAAV-Sur(wt) expressed high levels wild type survivin protein and contained fewer apoptotic cells relative to the PBS injected tumors (FIG. 5C). These results demonstrate that infection with rAAV-Sur-mut(Cys84Ala) induced apoptosis and mitotic catastrophe in vivo.

Synergetic Anti-tumor Effect of rAAV-Sur-Mut(Cys84Ala) and 5-FU in vivo

The combination effect of rAAV-Sur-Mut(Cys84Ala) and 5-FU on tumor growth was assayed. These tumors were injected in three sites with rAAV-Sur(wt), rAAV-eGFP or rAAV-Sur-Mut(Cys84Ala) at 5×10¹⁰ pfu/site. The mice were additionally injected i.p. with 50 mg/kg 5-FU for 5 days. rAAV-Sur-Mut(Cys84Ala) strongly enhanced the anti-tumor activity of 5-FU in this colon cancer xenograft model. Complete regression of pre-established tumor was found in mice treated with the combination of rAAV-Sur-Mut(Cys84Ala) and 5-FU in 6 out of 8 animals 4 weeks after treatment. In contrast, co-treatment with rAAV-eGFP and 5-FU did not potentiate the anti-tumor effect (Table 2). Furthermore, co-treatment with rAAV-Sur(wt) and 5-FU reduced the anti-tumor effect of 5-FU, relative to treatment with 5-FU alone. Tumor regression was attributed to elevated rates of apoptosis as assessed by TUNEL staining. Tumors injected with rAAV-Sur-Mut(Cys84Ala) in combination with 5-FU therapy contained more apoptotic cells. In contrast, tumors injected with rAAV-eGFP or rAAV-Sur(wt) and 5-FU showed fewer TUNEL-positive cells (FIG. 6A).

To investigate whether treatment with rAAV-Sur-Mut(Cys84Ala) and 5-FU was long lasting, we also assessed survival. Mice challenged with SW1116 died from tumor burden or were sacrificed when they reached experimental end-points (tumor size>than 2.5 cm³) when treated with vehicle buffer, rAAV-Sur(wt) and rAAV-eGFP (mean survival: buffer, 44.2±1.9 days; rAAV-Sur(wt), 41.6±1.6 days; rAAV-EGFP, 46±2.9 days). Although mice treated with 5-FU or rAAV-Sur-Mut(Cys84Ala) alone survived significantly longer than buffer-treated mice (P<0.0012), all eventually died (FIG. 6B). Notably, 4 out of 8 animals treated with the combination of rAAV-Sur-Mut(Cys84Ala) and 5-FU were still alive without any visible tumors and without clinical symptoms 86 days after treatment (FIG. 6B). Furthermore, all of the animals survived significantly longer than mice treated with 5-FU or rAAV-Sur-Mut(Cys84Ala) alone (P<0.0016). Tumor-bearing mice injected intratumorally with the combination of rAAV-Sur-Mut(Cys84Ala) and 5-FU showed no clinical symptoms. By contrast, mice treated with vehicle buffer, rAAV-eGFP, AAV-Sur(wt) or 5-FU alone developed severe symptoms related to tumor growth. Although further studies are necessary to better assess the effect of the combined treatment with rAAV-Sur-Mut(Cys84Ala) and 5-FU or other therapeutic drugs, these findings indicate that rAAV-Sur-Mut(Cys84Ala) and 5-FU improved survival in vivo.

TABLE 2
rAAV-Sur-Mut(Cys84Ala) triggers tumor regression in combination with 5-FU

| | | Number of mice tumor | | | |
|---|---|---|---|---|---|
| No. | Treatment | with regression | Tumor size (mm³) | Rate of inhibition | P |
| 1 | PBS Buffer | 0/4 | 1567.8 ± 139.3 | | |
| 2 | 5-FU | 0/4 | 606.3 ± 106.7 | 61.2% | 2 vs 1: P = 0.0016 |
| 3 | AAV-EGFP + 5-FU | 0/4 | 495.3 ± 93.9 | 68.4% | 3 vs 2: P = 0.283 |
| 4 | rAAV-Sur(wt) + 5-FU | 0/4 | 849.4 ± 136.7 | 45.1% | 4 vs 2: P = 0.047 |
| 5 | AAAV-Sur-Mut + 5-FU | 3/4 | 27 ± 13.6 | 98.2% | 5 vs 2: P = 0.0002 |
| | | | | | 5 vs 3: P = 0.0004 |
| SW1116 cells were injected into the left flank (s.c.) in female nude mice. Tumors were allowed to grow to approximately 100-150 mm³ volume, and injected with PBS, rAAV-eGFP or rAAV-Sur(wt) or rAAV-Sur-Mut in three sites (5 × 10¹⁰ particles | | | | | |
| # virus/site), in combinations of 5-FU i.p. injection (50 mg/kg of 5-FU on each of 5 days). Tumor volumes were measured every week after treatment. The experiments were repeated twice. Mean tumor volume, rate of inhibition and P values between the different treatment groups 1-5 are indicated. | | | | | |

rAAV-Sur-Mut(Cys84Ala) Induced Apoptosis in Endothelial Cells and Reduced Tumor Angiogenesis in vivo

To further investigate the effect of inhibition of survivin on tumor angiogenesis, expression of PECAM-1/CD31 and apoptosis of endothelial cells was assayed using an immunofluorescent endothelial cell marker (anti-CD31-red) and a fluorescent apoptosis marker (TUNEL-green). As shown in FIG. 7, microvessel density (MVD) in tumors injected with rAAV-Sur-Mut(Cys84Ala) was significantly reduced compared with those in tumors injected with rAAV-eGFP, rAAV-Sur(wt) and PBS (P<0.05; FIG. 7B). Using double-staining immunofluorescence, significant tumor cell (green) and endothelial cell (yellow) apoptosis were observed in tumors treated with rAAV-Sur-Mut(Cys84Ala) compared with those in tumor injected with rAAV-Sur(wt), rAAV-eGFP and PBS (FIG. 7B). In addition, endothelial cell apoptosis wasn't detected in tumor injected with rAAV-Sur(wt) compared with rAAV-eGFP. These results indicated that inhibition of survivin function reduced tumor angiogenesis in vivo, partly by inducing apoptosis of endothelial cells.

Discussion

This study shows that AAV mediated survivin Cys84Ala mutant induced spontaneous apoptosis and mitotic catastrophe in colon cancer both in vitro and in vivo, inhibited angiogenesis and tumor growth, and triggered the regression of established tumor when used in combination with 5-FU in vivo.

Survivin has attracted significant attention as a target for cancer therapy due to its differential expression in tumors versus normal tissues and its potential requirement for maintaining cancer cell viability.¹ The major advantages of AAV vectors include stable integration, low immunogenicity, long-term expression, and ability to infect both dividing and nondividing cells.^{26,27} An orally administered AAV vector was documented to result in persistent expression of a β-galactosidase reporter transgene in both colon epithelial and lamina propria cells.³⁶ Our results demonstrate that AAV efficiently transduced colon cancer in vitro and in vivo. The transduction rate in colon cancer cells is similar to that in glioma, breast and pancreatic cancer cells.^{31,39} Here, AAV-mediated Sur-Mut(Cys84Ala) expression was sufficient to directly inhibit proliferation and induce apoptosis in colon cancer. Consistently, transduction with rAAV-Sur-Mut(Cys84Ala) resulted in increased expression of mutant survivin protein, enhanced caspase 3 activity, as well as caspase-3 and PARP cleavage in colon cancer cells.

In addition to triggering apoptosis, we further confirmed that expression of Sur-Mut(Cys84Ala) mediated by AAV caused mitotic catastrophe in colon cancer cells in vitro and in vivo. Mitotic catastrophe is a form of cell death characterized by a significant increase in abnormal appearing nuclei that include multiple, multilobulated nuclei and abnormally large nuclei, as well as unresolved chromosomes bridges, a marker that the cell initiated but did not complete cytokinesis.⁴² Previous work showed that survivin Thr34Ala mutant induces apoptosis, but failed to cause cell cycle arrest or mitotic catastrophe in tumor cells.²⁴ However, a recent study showed that stable expression of survivin Thr34Ala mutant caused aberrant cytokinesis, not apoptosis.⁴³ Our results showed that rAAV-Sur-Mut(Cys84Ala) induced both apoptosis and mitotic catastrophe in colon cancer in vitro and in vivo. This finding has important implication for cancer therapy. An increase in the frequency of micronucleated tumor cells after radiotherapy and chemotherapy was suggested to be a positive prognostic marker of treatment response. Mitotic catastrophe in vivo usually leads to necrosis, thereby causing local inflammation that may intensify the anti-tumor effect.⁴² A previous report showed expression of survivin antisense or Cys84Ala mutant survivin led to increased anti-tumor cytotoxic T-lymphocytes and sensitized tumor cells to immunotherapy in vivo.²⁵ This may be in part attributable to mitotic catastrophe.

One important finding reported here was that targeting survivin appears to compromise endothelial cell viability and suppressed tumor angiogenesis in vivo. The expression of survivin is correlated with angiogenesis and/or shorter survival of patients with colorectal cancer.³⁵ In this study, besides induction of apoptosis and mitotic catastrophe, transduction with rAAV-Sur-Mut(Cys84Ala) was sufficient to directly induce apoptosis of endothelial cells and inhibit angiogenesis in the xenograft model. This was consistent with the results obtained in vitro in which survivin antisense treatment induced rapid regression of three-dimensional vascular capillary networks.¹⁷ Importantly, induction of survivin in endothelial cells is observed during the proliferative or remodeling phases of angiogenesis and is associated with resistance to apoptosis. In contrast, survivin is undetectable in quiescent endothelium in vitro and in vivo.¹⁴⁻¹⁶ and targeting survivin does not affect endothelial cell viability in quiescent endothelium.¹⁷ rAAV-Sur(wt) suppressed endothelial cell apoptosis in the xenografts relative to PBS or rAAV-eGFP, indicating that expression of survivin prevent endothelial cells from apoptosis in vivo. Inhibition of survivin may promote endothelial cells apoptosis during tumor angiogenesis, thus accelerating regression of newly formed blood vessels and reducing the incidence of metastatic disease. The selected expression of survivin in tumor cells and angiogenically stimulated endothelial cells may provide a high degree of specificity for potential survivin antagonists to enhance both anti-angiogenic and anti-neoplastic therapeutic strategies.

AAV vectors targeting regulators of apoptosis and/or angiogenesis inhibitors have been investigated for their potential suitability in cancer gene therapy alone and in combination with established chemotherapy regimes.^{44,45} A single intratumoral injection of AAV-Sur-Mut (Cya84Ala) was able to inhibit tumor growth and prolong survival in the nude mouse model. The reduction of tumor size was associated with decreased numbers of tumor vessels and increased apoptosis. These results support observations in other studies using rAAV vectors encoding angiostatin in an athymic mouse model bearing subcutaneous tumors.^{43,44} However, AAV-Sur-Mut(cys84Ala) therapy alone could not completely eradicate tumors. Therefore, we explored a combined approach using both AAV-mediated survivin mutant and chemotherapy. rAAV-Sur-Mut(Cys84Ala) significantly increased the sensitivity of colon cancer cells to classical chemotherapeutic drugs. rAAV-Sur-Mut(Cys84Ala) plus 5-FU triggered the regression of established tumors and significantly prolonged the survival of nude mice bearing colon cancer cell derived tumors. In contrast, AAV-Sur(wt) therapy reduced the anti-tumor effect of 5-FU. This study demonstrated a synergistic effect by of combined survivin gene therapy and chemotherapy.

References

1. Ambrosini G, et al., “A Novel Anti-Apoptosis Gene, Survivin, Expressed in Cancer and Lymphoma,” Nat. Med. (1997) 3, pp. 917-921.
2. Li F, et al., “Control of Apoptosis and Mitotic Spindle Checkpoint by Survivin,” Nature (1998) 396, pp. 580-584.
3. Giodini A, et al., “Regulation of Microtubule Stability and Mitotic Progression by Survivin” Cancer Res. (2002) 62, pp. 2462-2467.
4. Altieri, D C, “The Molecular Basis And Potential Role Of Survivin In Cancer Diagnosis And Therapy,” Trends Mol. Med. (2001) 7, pp. 542-547.
5. Tamm I, et al., “IAP-Family Protein Survivin Inhibits Caspase Activity and Apoptosis Induced by Fas (CD95), Bax, Caspases, and Anticancer Drugs,” Cancer Res. (1998) 58, pp. 5315-5320.
6. Grossman D, et al., “Transgenic Expression of Survivin in Keratinocytes Counteracts UVB-Induced Apoptosis and Cooperates with Loss of P53,” J. Clin. Invest. (2001) 108, pp. 991-999.
7. Suzuki A, et al., “Survivin Initiates Cell Cycle Entry by the Competitive Interaction with Cdk4/P16(INK4a) and Cdk2/Cyclin E Complex Activation,” Oncogene (2000) 19, pp. 3225-3234.
8. Allen S M, et al., “Survivin Expression in Mouse Skin Prevents Papilloma Regression and Promotes Chemical-Induced Tumor Progression,” Cancer Res. (2003) 63, pp. 567-572.
9. Swana H S, et al., “Tumor Content of the Anti-Apoptosis Molecule Survivin and Recurrence of Bladder Cancer” N. Engl. J. Med. (1999) 341, pp. 452-453.
10. Tanaka, K, et al., “Expression of Survivin and its Relationship to Loss of Apoptosis in Breast Carcinomas,” Clin. Cancer Res. (2000) 6, pp. 127-134.
11. Adida, C, et al., “Prognostic Significance of Survivin Expression in Diffuse Large B-Cell Lymphomas,” Blood (2000) 96, pp. 1921-1925.
12. Kato J, et al., “Expression of Survivin in Esophageal Cancer: Correlation with the Prognosis and Response to Chemotherapy,” Int. J. Cancer (2001) 95, pp. 92-95.
13. Altieri D C, “Validating Survivin as a Cancer Therapeutic Target” Nat. Rev. Cancer (2003) 3, pp. 46-54.
14. O'Connor D S, et al., “Control of Apoptosis During Angiogenesis by Survivin Expression in Endothelial Cells,” Am. J. Pathol. (2000) 156, pp. 393-398.
15. Tran J, et al., “Marked Induction of the IAP Family Anti-Apoptotic Proteins Survivin and XIAP by VEGF in Vascular Endothelial Cells,” Biochem. Biophys. Res. Commun. (1999) 264, pp. 781-788.
16. Papapetropoulos A, et al., “Angiopoietin-1 Inhibits Endothelial Cell Apoptosis via the Akt/Survivin Pathway,” J. Biol. Chem. (2000) 275, pp. 9102-9105.
17. Mesri M, et al., “Suppression of Vascular Endothelial Growth Factor-Mediated Endothelial Cell Protection by Survivin Targeting,” Am. J. Pathol. (2001) 158, pp. 1757-1765.
18. Li F, et al., “Pleiotropic Cell-Division Defects and Apoptosis Induced by Interference with Survivin Function,” Nat. Cell. Biol. (1999) 1, pp. 461-466.
19. Grossman D, et al., “Inhibition of Melanoma Tumor Growth In Vivo by Survivin Targeting,” Proc. Natl. Acad. Sci. USA (2001) 98, pp. 635-640.
20. Olie R A, et al., “A Novel Antisense Oligonucleotide Targeting Survivin Expression Induces Apoptosis and Sensitizes Lung Cancer Cells to Chemotherapy,” Cancer Res. (2000) 60, pp. 2805-2809.
21. Yamamoto, T., et al., “Downregulation of Survivin Expression by Induction of the Effector Cell Protease Receptor-1 Reduces Tumor Growth Potential and Results in an Increased Sensitivity to Anticancer Agents in Human Colon Cancer,” Eur. J. Cancer (2002) 38, pp. 2316-2324.
22. Wall N R, et al., “Suppression of Survivin Phosphorylation on Thr34 by Flavopiridol Enhances Tumor Cell Apoptosis,” Cancer Res. (2003) 63, pp. 230-235.
23. O'Connor D S, et al., Altieri D C, “A P34(Cdc2) Survival Checkpoint in Cancer” Cancer Cell (2002) 2, pp. 43-54.
24. Mesri M, et al., “Cancer Gene Therapy Using a Survivin Mutant Adenovirus,” J. Clin. Investig. (2001) 108, pp. 981-990.
25. Kanwar, J R, et al., “Effects of Survivin Antagonists on Growth of Established Tumors and B7-1 Immunogene Therapy,” J. Natl. Cancer Inst. (2001) 93, pp. 1541-1552.
26. Ponnazhagan S, et al., “Adeno-Associated Virus For Cancer Gene Therapy,” Cancer Res. (2001) 61, pp. 6313-6321.
27. Kaplitt M G, et al., “Long-Term Gene Expression and Phenotypic Correction Using Adeno-Associated Virus Vectors in the Mammalian Brain,” Nat. Genet. (1994) 8, pp. 148-154.
28. Wang B, et al., “Adeno-Associated Virus Vector Carrying Human Minidystrophin Genes Effectively Ameliorates Muscular Dystrophy in Mdx Mouse Model,” Proc. Natl. Acad. Sci. USA (2000) 97, pp. 13714-13719.
29. Kay M A, et al., “Evidence for Gene Transfer and Expression of Factor IX in Haemophilia B Patients Treated with an AAV Vector,” Nat. Genet. (2000) 24, pp. 257-261.
30. Nguyen J T, et al., “Adeno-Associated Virus-Mediated Delivery of Antiangiogenic Factors as an Antitumor Strategy,” Cancer Res. (1998) 58, pp. 5673-5677.
31. Enger P Q, et al., “Adeno-Associated Viral Vectors Penetrate Human Solid Tumor Tissue In Vivo More Effectively Than Adenoviral Vectors,” Human Gene Therapy (2002) 13, pp. 1115-1125.
32. Kawasaki H, et al., “Inhibition of Apoptosis by Survivin Predicts Shorter Survival Rates in Colorectal Cancer,” Cancer Res. (1998) 58, pp. 5071-5074.
33. Sarela A I, et al., “Immunohistochemical Detection of the Anti-Apoptosis Protein, Survivin, Predicts Survival After Curative Resection of Stage II Colorectal Carcinomas,” Ann. Surg. Oncol. (2001) 8, pp. 305-310.
34. Zhang T, et al., “Evidence That APC Regulates Survivin Expression: A Possible Mechanism Contributing to the Stem Cell Origin of Colon Cancer,” Cancer Res. (2001) 61, pp. 8664-8667.
35. Kawasaki H, et al., “Expression of Survivin Correlates with Apoptosis, Proliferation, and Angiogenesis During Human Colorectal Tumorigenesis,” Cancer (2001) 91, pp. 2026-2032.
36. During M J, et al., “An Oral Vaccine Against NMDAR1 with Efficacy in Experimental Stroke and Epilepsy,” Science (2000) 287, pp. 1453-1460.
37. Ho S N, et al., “Site-Directed Mutagenesis by Overlap Extension Using the Polymerase Chain Reaction,” Gene (1989) 77, pp. 51-9.
38. Xu R, et al., “Quantitative Comparison of Expression with Adeno-Associated Virus (AAV-2) Brain-Specific Gene Cassettes,” Gene Ther. (2001) 8, pp. 1323-1332.
39. Dumon K R, et al., “Fragile Histidine Triad Expression Delays Tumor Development and Induces Apoptosis in Human Pancreatic Cancer,” Cancer Res. (2001) 61, pp. 4827-4836.
40. Giatromanolaki A, et al., “Comparative Evaluation of Angiogenesis Assessment with Anti-Factor-VIII and Anti-CD31 Immunostaining in Non-Small Cell Lung Cancer,” Clin. Cancer Res. (1997) 3(12 Pt 1), pp. 2485-2492.
41. Baker C H, et al., “Blockade of Epidermal Growth Factor Receptor Signaling on Tumor Cells and Tumor-Associated Endothelial Cells for Therapy of Human Carcinomas,” Am. J. Pathol. (2002) 161, pp. 929-38.
42. Roninson I B, et al., “If Not Apoptosis, Then What? Treatment-Induced Senescence and Mitotic Catastrophe in Tumor Cells” Drug Resist Update (2001) 4, pp. 303-313.
43. Temme A, Rieger M, Reber F, Lindemann D, Weigle B, Diestelkoetter-Bachert P, Ehninger G, Tatsuka M, Terada Y, Rieber E P, “Localization, Dynamics, And Function Of Survivin Revealed By Expression Of Functional Survivin-dsred Fusion Proteins In The Living Cell” Mol Biol Cell (2003) 14, pp. 78-92.
44. Davidoff A M, et al., “Raav-Mediated Long-Term Liver-Generated Expression of an Angiogenesis Inhibitor Can Restrict Renal Tumor Growth in Mice,” Cancer Res. (2002) 62, pp. 3077-3083.
45. Ma H I, et al., “Suppression of Intracranial Human Glioma Growth After Intramuscular Administration of an Adeno-Associated Viral Vector Expressing Angiostatin,” Cancer Res. (2002) 62, pp. 756-763.

What is claimed is:

1. A vector comprising a rAAV-type 2 plasmid encoding mutant survivin (Cys84Ala).

2. A vector comprising a rAAV-type 2 plasmid encoding eGFP.

3. A composition comprising the vector of claim 1.

4. A composition comprising the vector of claim 2.

5. A pharmaceutical composition comprising the vector of claim 1 and a pharmaceutically acceptable carrier.

6. A method for inducing apoptosis in a cell comprising introducing into the cell the vector of claim 1.

7. The method of claim 6, wherein the cell is a cancer cell.

8. The method of claim 7, wherein the cancer cell is a colon cancer cell.

9. The method of claim 6, wherein the vector is introduced by transduction.

10. A method for inducing apoptosis in a cell comprising introducing into the cell the composition of claim 3.

11. The method of claim 10, wherein the cell is a cancer cell.

12. The method of claim 11, wherein the cancer cell is a colon cancer cell.

13. The method of claim 10, wherein the vector is introduced by transduction.

14. A method for inhibiting tumor cell growth in an animal afflicted with cancer, comprising introducing into a tumor cell the vector of claim 1.

15. The method of claim 14, wherein the tumor cell is a colon cancer cell, and the cancer is colon cancer.

16. The method of claim 14, wherein the vector is introduced into the animal by transduction.

17. A method for inhibiting tumor cell growth in an animal having a tumor comprising introducing into the tumor cell the composition of claim 3.

18. The method of claim 17, wherein the tumor cell is a colon cancer cell.

19. The method of claim 17, wherein the vector is introduced by transduction.

20. A method for treating a subject afflicted with colon cancer comprising administering to the subject a therapeutically effective amount of the vector of claim 1 in a pharmaceutically acceptable carrier.

21. The method of claim 20, wherein the vector transduces colon cancer cells.

22. The method of claim 20, wherein the subject is human.

23. The method of claim 20 further comprising administering a suitable chemotherapy agent.

24. The method of claim 23, wherein the chemotherapy agent is 5-FU or cisplatin.

25. A method for inhibiting the growth of a tumor in a subject afflicted with colon cancer comprising administering to the subject a therapeutically effective amount of the pharmaceutical composition of claim 5.

26. The method of claim 25, wherein the subject is human.

27. The method of claim 25 further comprising administering a suitable chemotherapy agent.

28. The method of claim 22, wherein the chemotherapy agent is 5-FU or cisplatin.
